(19) United States
(12) Patent Application Publication
(10) Pub. No.: US 2024/0299017 A1
(43) Pub. Date: Sep. 12, 2024

(54) Examination Scope With Tissue Sample Collection Brush

(71) Applicant: REGENTS OF THE UNIVERSITY OF MICHIGAN, Ann Arbor, MI (US)

(72) Inventors: Marilyn S. Filter, Saginaw, MI (US); Lyn Behnke, Tawas City, MI (US); Line Van Niewstadt, Ann Arbor, MI (US); Quamrul Mazumder, Ann Arbor, MI (US)

(21) Appl. No.: 18/271,031

(22) PCT Filed: Jan. 6, 2022
(86) PCT No.: PCT/US22/11387

§ 371 Date: Jul. 5, 2023

Related U.S. Application Data
(60) Provisional application No. 63/134,249, filed on Jan. 6, 2021.

(51) Int. Cl. A61B 10/04; A61B 1/00; A61B 1/04; A61B 1/06; A61B 1/07; A61B 1/303; A61B 1/31; A61B 10/02

(52) U.S. Cl. CPC A61B 10/04; A61B 1/00087; A61B 1/00096; A61B 1/00101; A61B 1/042; A61B 1/0669; A61B 1/07; A61B 1/0607; A61B 1/303; A61B 1/31; A61B 2010/0216

(57) ABSTRACT

An apparatus for conducting gynecological or proctological exams includes a sensor base having a proximal end and a distal end. The apparatus also includes a light source disposed within the sensor base and a camera disposed within the sensor base. Further, the apparatus includes a brush extending from the distal end of the sensor base and adapted to collect samples for testing.

### CROSS-REFERENCE TO RELATED APPLICATION

This application claims the benefit of the filing date of U.S. Provisional Patent Application No. 63/134,249, filed Jan. 6, 2021. The contents of U.S. Provisional Application No. 63/134,249 is hereby incorporated by reference in its entirety.

### FIELD OF THE INVENTION

This application relates generally to apparatuses and techniques for conducting perineal exams, and more particularly, to an apparatus for conducting gynecological and proctological exams and methods of using the same.

### BACKGROUND

Numerous medical examinations require the examination and tissue collection from inside a patient's body. For example, gynecological and proctological examinations include evaluation and tissue collection for a patient's rectum, vagina, and/or cervix. For example, typical gynecological examinations require a patient to sit with their legs elevated and spread in stirrups. A speculum is then used to mechanically stretch the vagina open and permit a medical practitioner to examine and collect tissue samples from the vagina and/or rectum, though require the practitioner to be in close proximity to the patient's genitalia.

The invasiveness and discomfort of these examinations cause many people to avoid these examinations. Avoiding such examinations can permit conditions, such as cancers or sexually transmitted diseases, to go undiagnosed. Leaving such conditions undiagnosed can be very detrimental to the health and life of a patient. Conducting routine examinations is the best method to determine early disease diagnosis or abnormalities. Accordingly, to improve patient compliance, there is a need for improved devices and methods that facilitate an atraumatic examination experience.

### SUMMARY

An apparatus for conducting perineal examinations, including gynecological and/or proctological exams, includes a sensor base having a proximal end and a distal end. The apparatus also includes a light source disposed within the sensor base and a camera disposed within the sensor base. Further, the apparatus includes a brush extending from the distal end of the sensor base and adapted to collect samples for testing.

An apparatus for conducting perineal examinations, including gynecological and/or proctological exams, includes a sensor base having a proximal end and a distal end. The sensor base includes a light source disposed at or near the distal end of the sensor base, a camera disposed within (at or near a distal end of) the sensor base, and a tissue swab, adapted to collect samples, extending from the distal end of the sensor base. The apparatus also includes and a protective sleeve disposed over the tissue swab.

An apparatus for conducting perineal examinations, including gynecological and/or proctological exams, including a sensor base having a proximal end and a distal end. A camera is disposed on the distal end of the sensor base. Also, the sensor base includes a photon source disposed on the sensor base and a photon transmitting element conveying photons from the photon source beyond the distal end of the sensor base

### BRIEF DESCRIPTION OF THE DRAWINGS

FIG. 1 is a side view of a sensor apparatus of a first embodiment of the present disclosure for gynecological and proctological examinations.

FIG. 2 is a perspective view of a sensor apparatus of a second embodiment of the present disclosure for gynecological and proctological examinations.

FIG. 3 is a perspective view of a sensor apparatus of a third embodiment of the present disclosure for gynecological and proctological examinations.

FIG. 4 is a side view of the sensor apparatus of any previous embodiment of the present disclosure for gynecological and proctological examinations including the brush head removed from the sensor apparatus.

FIG. 5 is a perspective view of a protective sleeve usable with any previous embodiment of the present disclosure for gynecological and proctological examinations.

FIG. 6 is a cross sectional view of the sensor apparatus of the first embodiment of the present disclosure for gynecological and proctological examinations.

FIG. 7a is a front view of a first example brush head of the present disclosure for gynecological and proctological examinations.

FIG. 7b is a front view of a second example brush head of the present disclosure for gynecological and proctological examinations.

FIG. 8 is a perspective view of a sensor system including a fourth embodiment of the sensor apparatus in accordance with the present disclosure for gynecological and proctological examinations.

FIG. 9 is a perspective view of the fifth embodiment of the sensor apparatus in accordance with the present disclosure for gynecological and proctological examinations.

FIG. 10a is a side view of a protective sleeve of a fifth embodiment of the sensor apparatus.

FIG. 10b is a side view of an inner sleeve of the fifth embodiment of the sensor apparatus.

FIG. 10c is a side view of an endoscope useable in the fifth embodiment of the sensor apparatus.

FIG. 10d is a side view of a cap of the fifth embodiment of the sensor apparatus.

FIG. 10e is a brush head useable in the fifth embodiment of the sensor apparatus.

FIG. 11 is a side view of the fifth embodiment of the sensor apparatus.

FIG. 12 is a side view of a sixth embodiment of the sensor apparatus in accordance with the present disclosure for gynecological and proctological examinations.

FIG. 13a is a front view of the sensor of either the fifth or sixth embodiment of the sensor apparatus.

FIG. 13b is a front view of the sensor having an alternative cap design.

FIG. 14 is a bottom view of the sensor of either the fifth or sixth embodiment of the sensor apparatus.

FIG. 15 is a perspective view of a first alternative brush useable in the fifth or sixth embodiment of the sensor apparatus.

FIG. 16 is a perspective view of a second alternative brush useable in the fifth or sixth embodiment of the sensor apparatus.

FIG. 17a is a side view of an additional embodiment of the sensor apparatus in accordance with the present disclosure.

FIG. 17b is a side view of the protective sleeve of FIG. 17a in accordance with the present disclosure.

### DETAILED DESCRIPTION

Current medical examination techniques for gynecological, rectal, and proctological examinations require patients to, typically, assume an uncomfortable examination position to provide the medical practitioner sufficient space to observe and operate the medical tools necessary for the examination. For example, gynecological exams often require female patients to take an uncomfortable and vulnerable position on their back with their legs up and spread in stirrups. Such positions can be particularly difficult for survivors of sexual assault.

Additionally, not all medical practitioners follow universal precautions in gynecological, rectal, and/or proctological examinations. Certain examinations require medical practitioners to place their face near examination area. As a result, during conventional examinations, the medical practitioner may get human discharge on their clothing, gloves, and/or face. Such contamination risks the health of the medical practitioner and other patients as well.

The sensor apparatus of the present disclosure improves the examination procedure for both the patient and the medical practitioner. First, the sensor apparatus allows the patient to undergo the examination from either a lateral or dorsal position without the use of stirrups. Additionally, the sensor apparatus minimizes the need to uncomfortably and mechanically stretch any patient body parts, such as the vagina with the use of a speculum. Further, the sensor apparatus improves the medical practitioner's examination by providing safer and more informational examinations and procedures. Images collected using the apparatus of the present disclosure may also be stored and transmitted for telehealth consultation or added to a patient's electronic medical records.

From the foregoing benefits, the sensor apparatus can revolutionize numerous medical fields. For example, the sensor apparatus can improve women's healthcare. Because many women avoid gynecological examinations due to discomfort and pain, the improved patient experience provided by the sensor apparatus can decrease the number of women who are not up to date on their critical examinations. Furthermore, the sensor apparatus can be used for preterm labor to avoid stimulation of the cervix or during emergency department visits involving vaginal complaints. Lastly, the sensor apparatus provides a better examination procedure that would more amenable to women who have been sexually assaulted to avoid feelings a further violation.

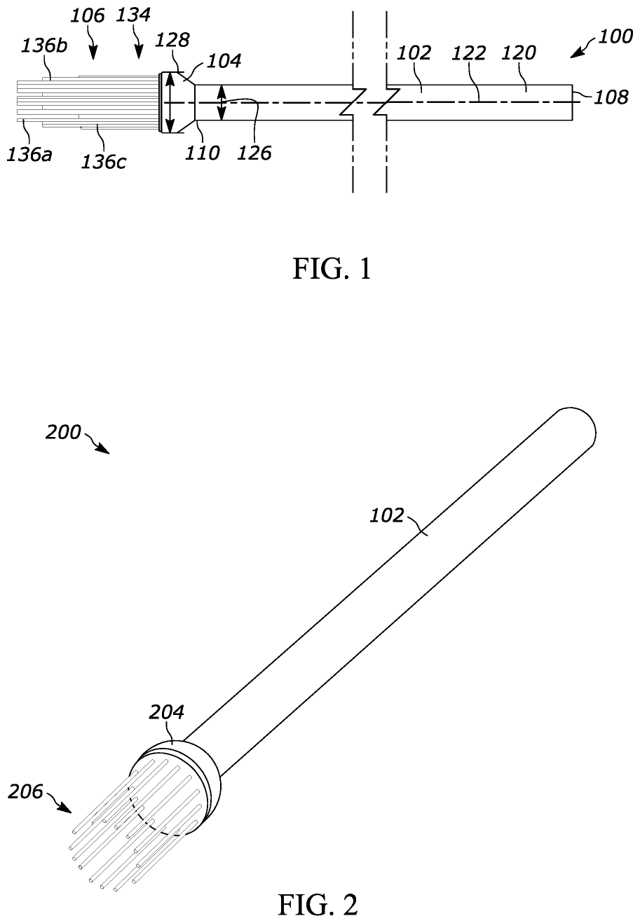

FIG. 1 is a sensor apparatus 100 for conducting gynecological and proctological examinations. The sensor apparatus 100 includes a sensor base 102, a brush head 104, and a brush 106. As illustrated in FIG. 1, the sensor base 102 includes a proximal end 108 and a distal end 110. Both the brush head 104 and the brush 106 are disposed on the distal end 110 of the sensor base 102. The sensor apparatus 100 can be made of various medical grade materials including polymers, fibers, silicon, metal, glass, etc.

The sensor base 102 defines a hollow cylindrical body 120 having a longitudinal axis 122 and an aperture (not shown) passing through the entire length of the sensor base 102. The cylindrical body 120 is designed to be sufficiently rigid to be inserted into a patient's body and thin enough to not cause discomfort to the patent. In some examples, the cylindrical body 120 is between approximately 200 millimeters (mm) and 300 mm along the longitudinal axis 122. Additionally, the cylindrical body 120 can have an outer diameter between about 8 mm and 15 mm while the inner diameter is between about 5 mm and 12 mm.

The brush head 104 is disposed on the distal end 110 of the sensor base 102. As illustrated in FIG. 1, the brush head 104 includes a first diameter 126 equal to the diameter of the sensor base 102 and a second diameter 128 larger than the first diameter 126. But the second diameter 128 can be smaller or the same size as the first diameter 126. Additionally, in some examples, the brush head 104 removably attaches to the distal end 110 of the sensor base 102. In such examples, the brush head 104 preferably includes a latching or locking mechanism to selectively secure the brush head 104 to the sensor base 102 but allowing a medical practitioner to disconnect the brush head 104 for testing (e.g., pathology). Alternatively, the brush head 104 could be integrally formed with the sensor base 102, but the brush head 104 could be configured to break off by incorporating a scoring line about the circumference of the sensor base 102 where the sensor base 102 joins the brush head 104.

The brush head 104 includes the brush 106 (sometimes referred to as a tissue swab). The brush 106 includes a plurality of optic fibers 134. Each of the plurality of optic fibers transmits light from a light source disposed within the sensor base 102. As a result, the sensor apparatus 100 can illuminate a surface near the brush 106 when directed towards that surface. For example, when placed in the vagina, the brush 106 can illuminate the cervix without use of a speculum or other mechanical stretching of the vagina. Additionally, the plurality of optic fibers 134 (e.g., the tissue swab) is adapted to collect tissue samples when brought into physical contact, when sufficient force is applied, with the tissues of a person. Each optic fiber of the plurality of optic fibers 134 can be between 0.3 mm and 1.5 mm in diameter to be sufficiently flexible to collect a tissue sample.

The brush head 104 is configured to facilitate the connection of the brush 106 to the sensor base 102 while still allowing the sensor base 102 to capture images or video. The brush head 104 is also configured to minimize discomfort for the patient. For example, the brush head has a diameter of approximately 12 mm and 25 mm. Further, the length of the brush head can be between about 5 mm and 15 mm, but at least part of the brush head 104 can be disposed over the sensor base 102. As illustrated, the brush head 104 includes two rows of optic fibers installed in concentric circles. The inner optic fibers can be manufactured to extend approximately 10 mm to 30 mm from the brush head 104 while the outer optic fibers can be extend about 10 mm to 20 mm from the brush head 104. Each of the inner and outer optic fibers comprise approximately 4 to 40 optic fibers (for a total of approximately 8 to 80 optic fibers in the plurality of optic fibers 134).

As illustrated in the example of FIG. 1, the brush head 104 includes a plurality of optic fibers 134 disposed in a shaped pattern. As illustrated, the plurality of optic fibers 134 extend beyond the brush head 104 at three distinct lengths. For example, optic fibers 136a, 136b, and 136c have different lengths that extend beyond the brush head 104 and provide the brush head 104 with a generally angled shape. Each optic fiber defines a photon transmitting element for conveying photons from the photon source beyond the distal end of the sensor base. The optic fibers are made from a flexible, transparent material, such as various types of polymers, ceramics, silica, or other materials suitable for the purpose of fiber-optic illumination.

FIG. 2 is a perspective view of a sensor apparatus 200 of a second embodiment of the present disclosure for gynecological and proctological examinations. The sensor apparatus 200 includes a sensor base 102 and brush head 204 and plurality of optic fibers 206. As illustrated in the example of FIG. 2, the plurality of optic fibers 206 are arranged in a different pattern than the plurality of optic fibers 134 as illustrated in FIG. 1. The plurality of optic fibers 134, 206 can be designed in a variety of patterns and three-dimensional shapes. As illustrated in FIG. 2, the plurality of optic fibers 206 are disposed at a single length extending beyond the distal end of the sensor base 102.

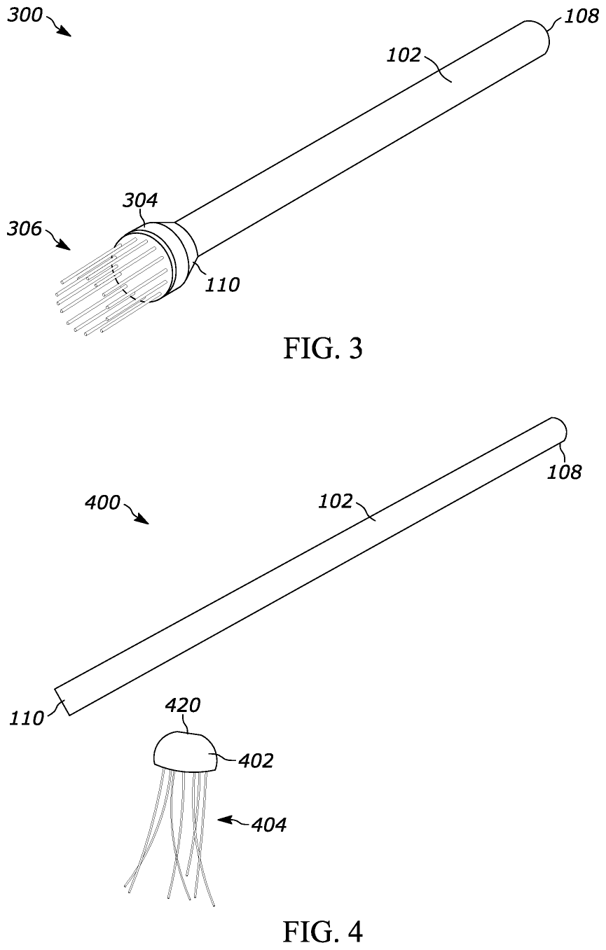

FIG. 3 is a perspective view of a sensor apparatus 300 of a third embodiment of the present disclosure for gynecological and proctological examinations. The sensor apparatus 300 includes a sensor base 102 and a brush head 304 having a plurality of optic fibers 306. As illustrated in the example of FIG. 3, the plurality of optic fibers 306 are arranged in a different pattern than the plurality of optic fibers 134 as illustrated in FIG. 1 or the plurality of optic fibers 206 as illustrated in FIG. 2. As illustrated in FIG. 3, the plurality of optic fibers 306 are arranged in a circular pattern and have alternating lengths extending beyond the distal end of the sensor base 102. It should be understood that the arrangement pattern is by way of example only. Additionally, the angle of the optic fibers with respect to the brush head 104/304 is not restricted to any particular angle.

FIG. 4 is a side view of the sensor apparatus 400 of any previous embodiment of the present disclosure for gynecological and proctological examinations including a sensor base 102 and a brush head 402 removed from the sensor base 102. Additionally, the brush 404 is disposed on a brush head removably attached to the distal end of the sensor base. In some examples, the brush head 402 includes a latching mechanism 420. The latching mechanism 420 is designed to fully secure the brush head 402 to the sensor base 102 when the sensor apparatus 400 is being used to collect tissue samples but easily removed from the sensor base 102 after the tissue sample is collected and the sensor apparatus 400 is removed from the patient. For example, after the sensor apparatus 400 is removed from a patient, the medical practitioner can disconnect the latching mechanism 420, causing the brush head 402 to no longer be disposed on the sensor base 102. The latching mechanism 420 can be any latching mechanism or other attachment mechanism known in the art to temporarily secure a cap to a cylinder. Because the brush head 402 is detachable from the sensor base 102, the sensor base 102, including a camera and light source (such as an endoscope), can be reused in future examinations after appropriate cleaning and sanitizing.

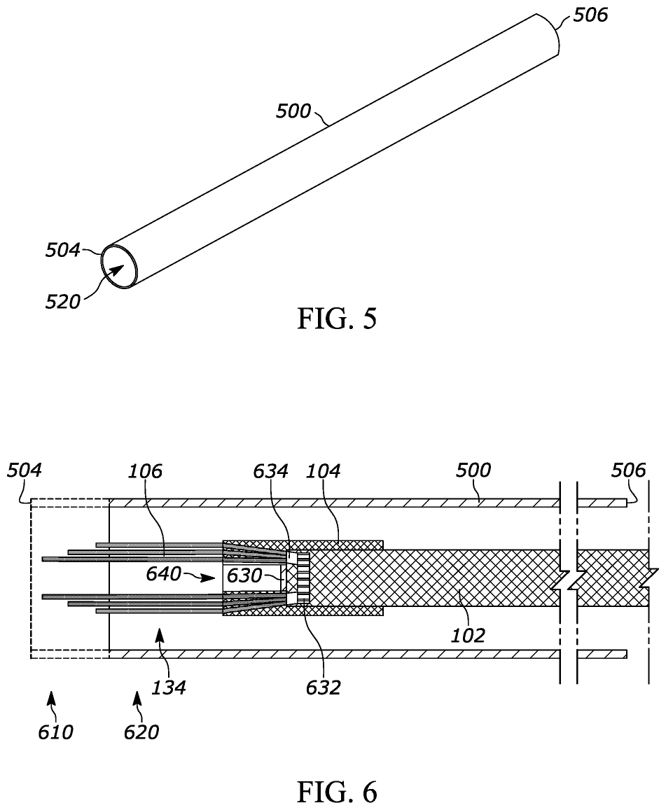

FIG. 5 is a perspective view of a protective sleeve 500 usable with any previous embodiment of the present disclosure for gynecological, proctological, and perineal examinations. The protective sleeve 500 includes a distal end 504, a proximal end 506, and a hollow aperture 520 disposed through the protective sleeve 500. The hollow aperture 520 is adapted to receive a sensor base, brush head, and brush to receive the sensor base, brush head, and brush, the hollow aperture needs to have a diameter between 8 millimeters (mm) and 25 mm. In some examples, the protective sleeve 500 is between 200 mm and 300 mm long and is longer than a sensor base. However, when the sensor base includes the brush head and brush, the brush can collect tissue samples beyond the distal end 504 of the protective sleeve 500. In other examples, the protective sleeve 500 is the same length, shorter, or longer than the sensor base.

In use, the protective sleeve 500 is inserted into the patient first, or the protective sleeve 500 and the sensor apparatus can be inserted into the patient as a single unit. The distal end 504 is inserted into the patient (e.g., inserted into the vagina) and the proximal end 506 remains disposed outside the patient's body. The protective sleeve 500 can be gently inserted into the patient's body until the protective sleeve 500 is disposed in a vicinity of the desired location. As a result, a medical practitioner can insert a sensor apparatus (of any of the embodiments of the disclosure) through the hollow aperture 520 at the proximal end 506. In some examples, the sensor apparatus can be inserted at the same time as the protective sleeve 500. With the assistance of the camera and light source (e.g., an endoscope), the sensor apparatus and protective sleeve can be used to collect a tissue sample of only the target location (e.g., a cervix). After the tissue sample is collected the sensor apparatus and the protective sleeve can be removed from the patient's body. After the tissue sample is collected, the sensor apparatus and protective sleeve may be used to visually inspect surrounding tissue during removal from the patient's body in real time, or to collect and store images for later study.

Because the protective sleeve 500 does not require mechanical stretching of the patient's vagina or rectum and inspection is conducted with a camera, the patient is not required to remain in an uncomfortable position (e.g., on their back with their legs in stirrups). Using the sensor apparatus of the present invention, the patient can undergo examination laying comfortably on the examination table, for example, on their side. Additionally, with the aid of the camera and proper training (e.g., a 30 minute training session) this sensor apparatus allows the medical practitioner to view the procedure via a screen, such as on a smartphone, tablet, or monitor in communication with the scope.

FIG. 6 is a cross sectional view of the sensor apparatus 100 of the first embodiment of the present disclosure for gynecological and proctological examinations. The sensor apparatus 100 includes a sensor base 102, a brush head 104, a brush 106, and a protective sleeve 500. During use, the protective sleeve 500 includes a distal end 504, inserted into the patient first, allowing the sensor apparatus 100 to be inserted into the proximal end 506. As illustrated in FIG. 6, the sensor base 102 is able to extend beyond the proximal end 506 of the protective sleeve 500.

The protective sleeve 500 is axially movable relative the brush head 104. During operation, the protective sleeve 500 is retractable, relative the sensor base 102, from a shielded position 610 disposed over, at least, the distal end 110 of the sensor base 102 and a retracted position 620 exposing the brush 106, the brush head 104, or the distal end 110 of the sensor base 102. The retracted position exposes a sufficient length of the brush 106 to provide light to the targeted area and also collect tissue samples via the plurality of optic fibers 134. Alternatively, the protective sleeve 500 is considered stationary and the sensor base 102 moves from a shielded position 610 to expose the plurality of optic fibers 106. As a result, during insertion of the protective sleeve 500 and the sensor base 102, the plurality of optic fibers 134 are inhibited from touching anything other than the desired body part. Additionally, the plurality of optic fibers 134 are protected from breaking during insertion.

In the illustrated example of FIG. 6, the inner diameter of the protective sleeve 500 is considerably greater than the outer diameter of the brush head 104. In other examples, the inner diameter of the protective sleeve 500 is approximate the outer diameter of the brush head 104. For example, the inner diameter of the protective sleeve could be up to around 20 millimeters (mm) and as small as 8 mm. The sensor base 102 and/or cap can have an outer diameter between approximately 8 mm and 20 mm.

Additionally, the sensor base 102 includes a camera 630 and a light source 632. The camera 630 is disposed on the distal end 110 of the sensor base 102. Additionally, as illustrated, the light source 632 (sometimes referred to as a photon emitter) is disposed within the sensor base 102. The sensor base 102 additionally includes channels 634 for transmitting light (e.g., photons) to the plurality of optic fibers 134. As a result, the light source 632 is able to transmit light from the light source, around the camera 632 and through the brush head 104 to illuminate a surface of an internal body part (e.g., a cervix). In some examples, the sensor base may include a plurality of light sources (e.g., approximately six light sources) disposed circumferentially within the sensor base 102 and may additionally include adjustable light intensity. Further, the camera 630 can be a high definition borescope or other high definition video camera disposed in an aperture 640 of the brush head 104. As a result, the camera 630 can obtain videos or still images beyond the distal end. For example, the camera 630 can be configured to produce adjustable resolution videos or still images having resolutions up to or greater than 1920 pixels by 1080 pixels. The camera 630 and the light source 632 may be both attached to an output or a semi-rigid snake capable for power and data transmission.

Additionally, the plurality of optic fibers 134 extend beyond the brush head 104 to both collect tissue samples and illuminate a target area. The plurality of optic fibers 134 can have a diameter between about 0.3 mm and 1.5 mm and can extend approximately 10 mm to 20 mm from the brush head 104 and can include optic fibers of various lengths. It is preferred that the plurality of optic fibers 134 extend straight out from the brush head 104. Accordingly, the plurality of optic fibers must have a minimum stiffness to direct light (e.g., photons) at a target location but must be sufficiently pliable to collect tissue samples effectively. Also, the optic fibers cannot be too stiff, because overly stiff optic fibers are more likely to break under stress. One way to ensure an appropriate amount of stiffness of the plurality of optic fibers is to provide a stiffening component such as an adhesive. In some examples, the adhesive is applied along the entire length of the plurality of optic fibers 134, while in other examples, the adhesive is selectively placed and only partially covers the length of the plurality of optic fibers 134.

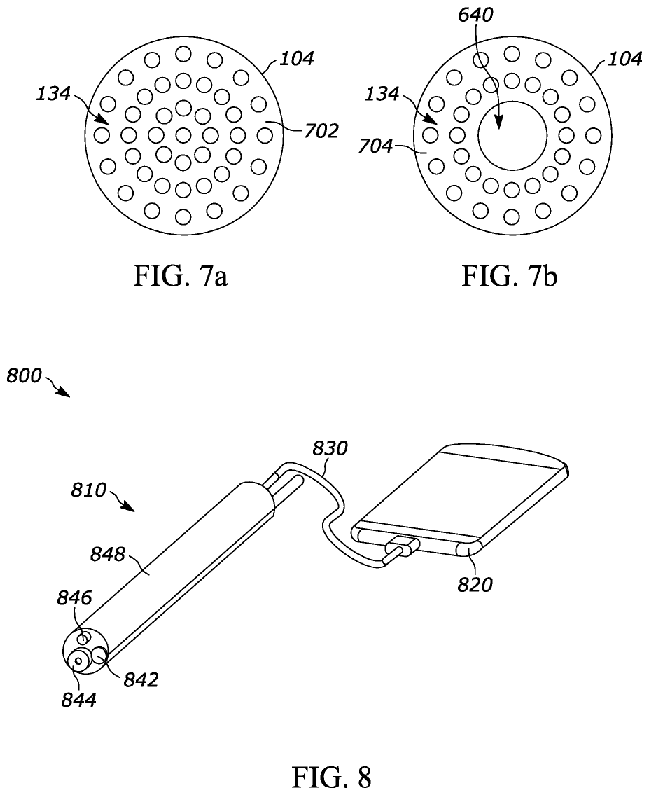

FIGS. 7a and 7b include alternative examples of brush heads. FIG. 7a includes a first brush head face 702 having equally distributed optic fibers and FIG. 7b includes a second brush head face 704 having optic fibers equally distributed about an annulus 640. Any configuration of optic fibers on the brush head face is considered in accordance with the teachings of the present disclosure.

FIG. 8 is a perspective view of a sensor system 800 including a fourth embodiment of the sensor apparatus 810 in accordance with the present disclosure for gynecological and proctological examinations. In the sensor system 800, the sensor apparatus 810 is connected to a display 820 via a cable 830. Further, the sensor apparatus 810 includes a sensor base 842, a camera 844, and a light source 846 all disposed on and within a protective sleeve 848.

In contrast to the sensor apparatus 100 of FIG. 1, the camera 844 and the light source 846 are not disposed within the sensor base 842. The camera 844 and the light source 846 are disposed adjacent the sensor base 842. As a result, the sensor base 842 cannot be equipped with a brush head to cover and protect the camera and light source. Additionally, because the light source is not covered by a brush head, the sensor base 842 does not rely on a plurality of optic fibers to illuminate a target area.

Additionally, the sensor apparatus 810 is electronically connected to a display 820 via a cable 830 connected to an output (not shown) on the sensor apparatus 810. As a result, still images and/or video captured by the camera 844 can be displayed on the display 820 to assist a medical practitioner in conducting an examination. The sensor apparatus 810 transmits the information obtained by the camera 844 via the cable 830. The cable 830 could be any type of USB cable, HDMI cable, DisplayPort, proprietary cable connection, or other cable adapted to transmit images generated from the camera. Further, in lieu of a cable 830, the sensor apparatus 810 can transmit the camera data via wireless technology, such as Bluetooth. While not shown, each of the sensor apparatus 100, 200, 300, 400, 800, 900, 1200 (sensor apparatus 900 and 1200 discussed in greater detail below) can include an output for transmitting camera data to a display. In each of the sensor apparatus 100, 200, 300, 400, 800, 900, 1200 the output can be disposed on the end opposite the brush to facilitate passing the sensor base through the protective sleeve, but can be disposed anywhere on the sensor apparatus 100, 200, 300, 400, 800, 900, 1200.

As illustrated in the example of FIG. 8, the display 820 is a mobile phone. For example, the sensor apparatus can be configured to work with any operating system, including Android 5.0 (and newer), iPhone iOS 9 (and newer), and other mobile phone and tablet operating systems. However, in other examples, the display could include a computer monitor, computer tablet, or other proprietary monitor. Additionally, in some examples, video or still images may be saved and stored in memory for later evaluation by a medical professional or for review and explanation to the patient. Accordingly, any of the sensor apparatus 100, 200, 300, 400, 800, 900, 1200 may include an internal power source, processor, and/or memory or may rely on an external power source, processor, and/or memory. The camera 844 (or any of the cameras of the other embodiments) could be a borescope capable of capturing high definition video.

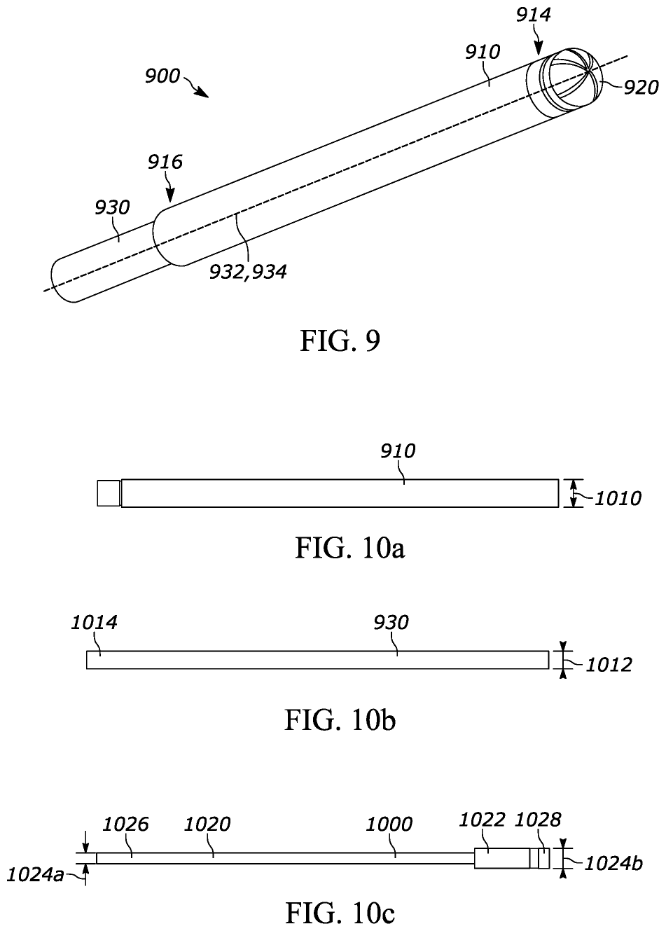

FIG. 9 is a perspective view of the fifth embodiment of the sensor apparatus 900 for gynecological and proctological examinations. The sensor apparatus 900 includes a protective sleeve 910, a cap 920, and an inner sleeve 930. In some embodiments, the cap 920 is integral with the protective sleeve 910 while in other embodiments the cap 920 is secured to the protective sleeve 910. Additionally, the protective sleeve 910 defines a longitudinal axis 932 and the inner sleeve 930 defines a longitudinal axis 934. In the illustrated embodiment of FIG. 9, the protective sleeve 910 and the inner sleeve 930 are coaxial, however in other examples, the longitudinal axis 932 and the longitudinal axis 934 may be parallel.

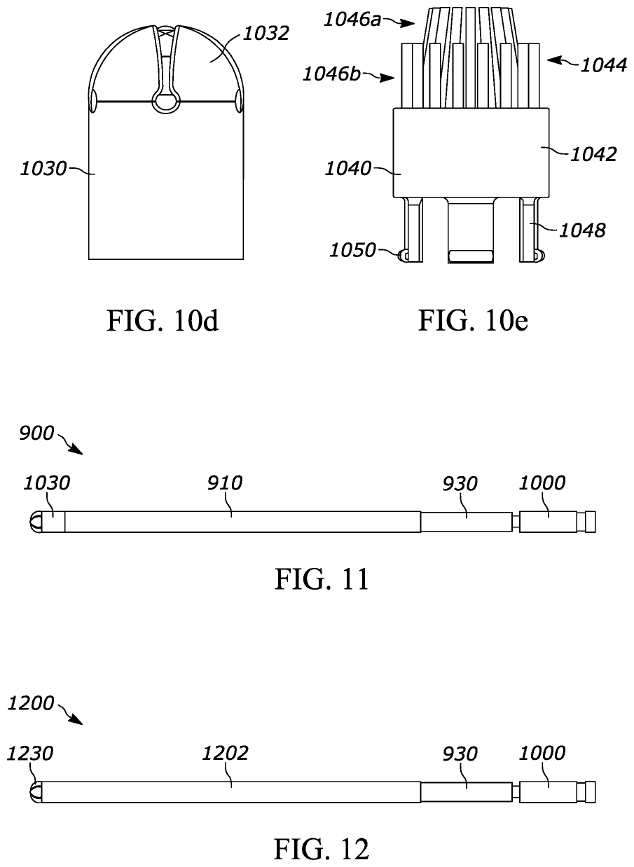

FIGS. 10a, 10b, and 10c are side views of the protective sleeve 910, the inner sleeve 930, and an endoscope 1000. In the present example, the inner sleeve 930 and the endoscope 1000 constitute the sensor base. The protective sleeve 910 is preferably made of a flexible material that resists cracking or breaking and mucosal compatible. In various examples, the protective sleeve 910 has an outer diameter 1010 of approximately 15 millimeters (mm). But the outer diameter 1010 of the protective sleeve 910 could be between 12 and 18 mm. The inner sleeve 930 defines an outer diameter 1012 that is configured to be clearance fit with the protective sleeve 910. The inner sleeve 930 may have a slide fit, running fit, easy slide fit, or loose running fit with the protective sleeve 910. For example, the ease slide fit between the protective sleeve 910 and the inner sleeve 930 provides only a small clearance between the protective sleeve 910 and the inner sleeve 930. In such an example, the inner sleeve 930 is configured to be stabilized by an inner surface of the protective sleeve 910, but the inner surface of the protective sleeve 910 is not so small as to cause excessive friction when the inner sleeve 930 is pushed through the protective sleeve 910. In some embodiments, the inner sleeve 930 and/or the endoscope 1000 incorporates a light source (not shown). In such embodiments, the light source transmits light to a brush head (illustrated in FIGS. 10e, 15, and 16) disposed on a distal end 1014 of the inner sleeve 930. Accordingly, a light source disposed in the inner sleeve 930 and/or the endoscope 1000 operates similarly to the light source 632 described in connection with FIG. 6.

As shown in FIG. 10c, the endoscope 1000 includes a sensor portion 1020 (e.g., a camera) at a distal end of the endoscope 1000 and a handle portion 1022 at a proximal end of the endoscope 1000. The sensor base includes a camera and a light source (not shown, but similar to the camera 630 and the light source 632). In some examples, the endoscope 1000 can be reusable, after appropriate cleaning and sanitization. In other examples, the endoscope 1000 is disposable and a new endoscope 1000 is used for each patient. As shown, the sensor portion 1020 defines a first diameter 1024a and a second diameter 1024b. The first diameter is disposed at a distal end 1026 of the sensor portion 1020 and the second diameter 1024b is disposed at a proximal end 1028. As shown, the second diameter 1028 is greater than the first diameter 1026. As illustrated in FIG. 14, the second diameter 1028 of the endoscope 1000 is less than the outer diameter 1010, but is greater than the outer diameter 1012 of the inner sleeve 930.

FIG. 10d is a side view of the cap 1030 having a at least one flap 1032. The flaps 1032 of the fifth embodiment could be pliable or semi-pliable material. The material used to manufacture the flaps 1032 could be made of a medical grade material, including a flexible plastic, a rubber, a synthetic polymer, or a natural flexible material. In a preferred embodiment, the flaps 1032 would be stiff enough to protect a brush head 1040 but also open up, without damaging the brush head, when the inner sleeve 930 is pushed through the protective sleeve 910.

FIG. 10e illustrates a brush head 1040 attachable to the inner sleeve 930 of FIG. 10b. The brush head 1040 is configured to facilitate the connection of the brush to the inner sleeve 930 while still allowing the endoscope 1000 to capture images or video. The brush head 1040 includes a hollow, cylindrical body 1042. The brush head 1040 further includes the brush having a plurality of optic fibers 1044 (sometimes referred to as a brush or tissue swab) capable of transmitting light from a light source. In some examples, the light source is placed in the inner sleeve 930 adjacent or proximate the brush head 1040 and the plurality of optic fibers 1044. The optic fibers are made from a flexible, transparent material, such as various types of polymers, ceramics, silica, or other materials suitable for the purpose of fiber-optic illumination. Each optic fiber of the plurality of optic fibers 1044 can be between 0.3 mm and 1.5 mm in diameter to be sufficiently flexible to collect a tissue sample. Additionally, each optic fiber of the plurality of optic fibers 1044 can be manufactured to extend approximately 10 mm to 40 mm from the brush head 1040.

As illustrated in FIG. 10e, the plurality of optic fibers 1044 include a first set of optic fibers 1046a and a second set of optic fibers 1046b. In the example, the first set of optic fibers 1046a is disposed within the second set of optic fibers 1046b, but the first set of optic fibers 1046a is longer than the second set of optic fibers 1046b. The arrangement of the plurality of optic fibers 1044 could be any arrangement, as illustrated in FIGS. 1, 2, 3, 6, 7a, 7b, 15, and 16.

Additionally, the brush head 1040 is configured to be selectively coupled to the inner sleeve 930. As illustrated the brush head 1040 includes at least one flexible arm 1048. Each flexible arm 1048 includes a protuberance 1050. The protuberance 1050 can correspond with an aperture or depression inside the inner sleeve 930 to secure the brush head 1040 to the inner sleeve 930. Alternatively, the brush head 1040 could be secured to the inner sleeve 930 by another means. For example, the brush head 1040 could be integral with the inner sleeve 930, and selectively coupled to the inner sleeve 930 by including a scoring line and/or perforation that allows the brush head to be snapped off the inner sleeve. The brush head 1040 could be selectively coupled to the inner sleeve by any method known in the art.

FIG. 11 is a side view of the sensor apparatus 900. As illustrated, the cap 1030 is disposed on the protective sleeve 910. Additionally, the inner sleeve 930 is disposed in the protective sleeve 910 and the endoscope 1000 is also disposed within both the protective sleeve 910 and the inner sleeve 930. Alternatively, FIG. 12 illustrates a sixth embodiment of the sensor apparatus 1200 for gynecological and proctological examinations. The sensor apparatus 1200 includes an protective sleeve 1202, the inner sleeve 930, and the endoscope 1000. In contrast to the sensor apparatus 900, the protective sleeve 1202 of the sensor apparatus 1200 includes an integral cap.

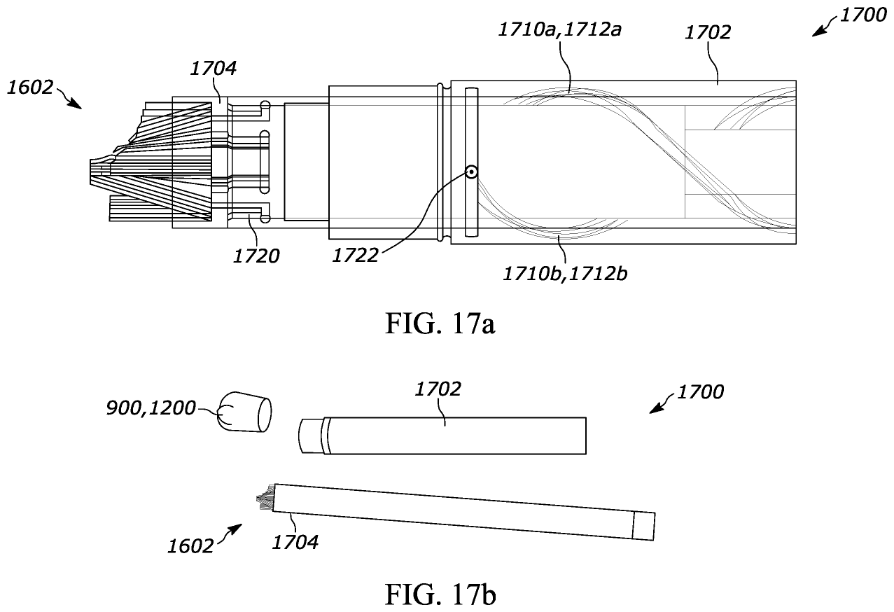

In use, the sensor apparatus 900, 122 is inserted into the patient, including the protective sleeve 910, 1202, the inner sleeve 930, and the endoscope 1000. In some uses, the protective sleeve 910, 1202 can be inserted into the patient before the inner sleeve 930 and the endoscope 1000. The cap 1030 is inserted into the patient first (e.g., inserted into the vagina). The protective sleeve 910, 1202 can be gently inserted into the patient's body until the protective sleeve 910, 1202 is disposed in a vicinity of the desired location. As a result, a medical practitioner can push the inner sleeve 930, including a brush head (e.g., the brush head 1040) to observe the desired location and collect a tissue sample. In some examples, the inner sleeve is controlled by hand, while in other examples, actuation of the inner sleeve relative the protective sleeve is controlled by a screw-mechanism (shown in greater detail in FIG. 17). With the assistance of the camera and light source (e.g., an endoscope), the sensor apparatus 900, 1200 can be used to collect a tissue sample of only the target location (e.g., a cervix). After the tissue sample is collected the sensor apparatus 900, 1200 can be removed from the patient's body. Also, the sensor apparatus 900, 1200 may be used to visually inspect surrounding tissue during removal from the patient's body in real time, or to collect and store images for later study. After a tissue sample is collected, the brush head is removed from the sensor apparatus and the tissue samples collected for study or other laboratory processes.

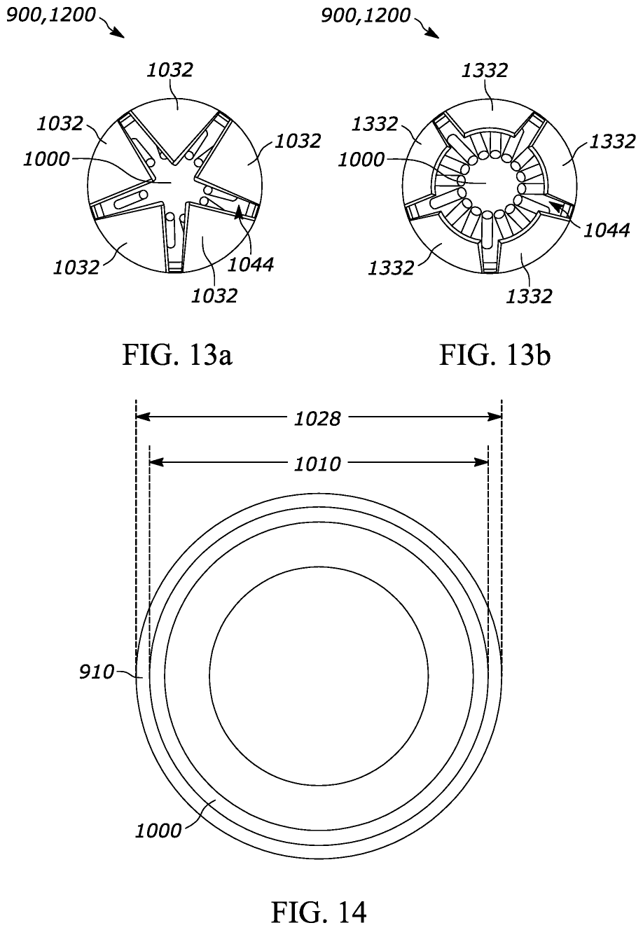

FIGS. 13a and 13b illustrate a front view of the sensor apparatus 900 or the sensor apparatus 1200. As shown, the sensor apparatus 900, 1200 includes five flaps 1032. In other examples, the cap 1030 or protective sleeve 1202 may include more or fewer flaps 1032. As shown, the plurality of optical fibers 1044 is protected by the cap 1030 or the protective sleeve 1202. When the inner sleeve 930 is pushed towards the flaps 1032, the inner sleeve pushes the flaps 1032 open allowing the plurality of optical fibers 1044 to exit the protective sleeve undamaged.

Both the cap 1030 of FIG. 13a and the cap 1330 of FIG. 13b includes five flaps 1032, 1332. The flaps 1332 are trapezoidal in shape in contrast to the triangular shape of flaps 1032. The trapezoidal shape of flaps 1332 still provide protection of the brush head 1040 but require less flexibility than the flaps 1032, because the flaps 1032 need to bend more to get out of the way of the brush head 1040.

FIG. 14 is a bottom view of the sensor apparatus 900, 1200. As shown, the outer diameter 1010 of the protective sleeve 910 is greater than the second diameter 1028 of the endoscope 1000. In some embodiments, the outer diameter 1010 of the protective sleeve 910 could be less than the second diameter 1028 of the endoscope 910.

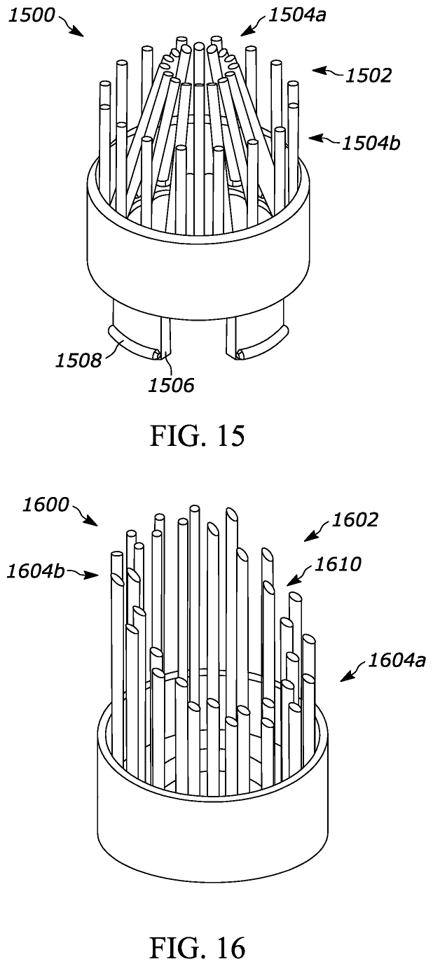

FIG. 15 is a first brush head 1500 useable with any sensor apparatus in accordance with the teaching of this disclosure. The first example brush head 1500, also illustrated in FIG. 10e, includes a plurality of optical fibers 1502 including a first set of optical fibers 1504a and a second set of optical fibers 1504b. The first set of optical fibers 1504a is longer than the second set of optical fibers 1504b, and also angled inwardly while the second set of optical fibers 1504b extend directly out of the brush ahead 1500. As shown in FIG. 10e, the brush head 1500 includes tabs 1506 and protuberances 1508. As a result, the brush head 1500 can be selectively coupled to the inner sleeve 930 if the inner sleeve includes corresponding apertures (not shown) to receive the protuberances 1508.

FIG. 16 is a second brush head 1600 useable with any sensor apparatus in accordance with the teachings of this disclosure. The second brush head 1600 includes a plurality of optical fibers 1602 disposed in an annular arrangement. The plurality of optical fibers 1602 is arranged such that the plurality of optical fibers are shorter in a first region 1604a of the brush head 1600 and longer in a second region of the brush head 1600. As illustrated, the length of the plurality of optical fibers 1602 transitions from the length of the first region 1604a to the length of the second region 1604b. The second brush head 1600 does not include tabs or protuberances, but may be integral with the inner sleeve 930. In such an embodiment, the brush head 1600 can be snapped off from the inner sleeve 930 if a scoring line is disposed between the brush head 1600 and the inner sleeve 930. In one example, the scoring line could be disposed approximately 2 centimeters (cm) from the edge of the brush head 1600. Alternatively, any known method of splitting an integral part can be utilized to separate the brush head 1600 from the inner sleeve 930. Because the plurality of optical fibers 1602 are disposed in an annular arrangement, light from a light source can also pass through an aperture 1610 of the annular arrangement.

FIGS. 17a and 17b are an example screw actuation mechanism 1700 for controlling the insertion of the brush head 1602 relative the protective sleeve 1702. As shown, the protective sleeve 1702 and the inner sleeve 1704 can include corresponding helical grooves 1710a, 1710b and rails 1712a, 1712b. As shown, when the inner sleeve 1704 is rotated, the grooves and rails 1710a, 1710b, 1712a, 1712b can cause the inner sleeve 1704 to move axially relative the protective sleeve 1702. While illustrated with two pairs of helical grooves and rails 1710a, 1710b, 1712a, 1712b, the screw actuation mechanism 1700 can have more or fewer pairs of grooves and rails (e.g., one pair, three pairs, four pairs). Because the brush head 1602 is axially actuated in response to the rotation of the screw actuation mechanism 1700, a medical professional is better able to control the exact position of the brush head 1602 relative the protective sleeve 1702. As shown in FIG. 17, the brush head 1602 is connected to the inner sleeve 1704 with tabs 1720, however, in other examples the brush head 1602 is integral with the inner sleeve 1704 but includes a scoring line to aide in snapping the brush head 1602 off the inner sleeve 1704.

Alternatively, the protective sleeve 1702 could include helical grooves 1710a, 1710b and the inner sleeve 1704 can include a protrusions 1722 configured to fit within the helical grooves 1710a, 1710b. As a result, the protrusions 1722 can pass through the helical grooves 1710a, 1710b and provide axial control of the inner sleeve 1704 relative the protective sleeve 1702.

In accordance with the teachings of the present disclosure, the brush heads 1500 and 1600 could have alternative configurations or designs. For example, each of the brush heads 1500 and 1600 could have more or fewer optical fibers. The brush heads 1500, 1600 could utilize any latching or locking mechanism to temporarily secure the brush heads 1500, 1600 to the inner sleeve 930, such as the locking mechanism 420. For example, the brush head 1600 could include tabs and protuberances while the brush head 1500 could be made integral with the inner sleeve 930. Alternatively, the inner sleeve 930 could have tabs and protuberances that are received in apertures or recesses in the brush head.

1. An apparatus for conducting gynecological or proctological exams, comprising:
a sensor base having a proximal end and a distal end;
a light source disposed within the sensor base;
a camera disposed within sensor base; and
a brush extending from the distal end of the sensor base and adapted to collect samples for testing.

2.-4. (canceled)

5. The apparatus of claim 1, wherein light from the light source passes through an aperture in the brush.

6. The apparatus of claim 1, wherein the brush is disposed on a brush head removably attached to the distal end of the sensor base.

7. The apparatus of claim 6, wherein the brush head is at least partly disposed over the light source and the camera.

8. The apparatus of claim 1, further including a protective sleeve disposed over the brush and at least a portion of the sensor base.

9. The apparatus of claim 8, wherein the protective sleeve is removably attached to the sensor base.

10. The apparatus of claim 1, wherein the camera is electronically connected to an output adapted to transmit images generated from the camera.

11. (canceled)

12. The apparatus of claim 1, in combination with a power source and a processor.

13. An apparatus for conducting gynecological or proctological exams, comprising:
a sensor base having a proximal end and a distal end, the sensor base including:
a light source disposed on the distal end of the sensor base;
a camera disposed on the distal end of the sensor base; and
a tissue swab, adapted to collect samples, disposed on the distal end of the sensor base; and
a protective sleeve disposed over the tissue swab.

14. The apparatus of claim 13, wherein the tissue swab is disposed on a brush head removably attached to the distal end of the sensor base.

15. The apparatus of claim 14, wherein the brush head includes a scoring line configured to snap the brush head off the sensor base.

16. The apparatus of claim 14, wherein the brush head is at least partly disposed over the light source and the camera.

17. The apparatus of claim 13, further including an output, electronically connected to the camera, adapted to transmit images generated from the camera.

18. (canceled)

19. The apparatus of claim 13, wherein the protective sleeve is removably attached to the tissue swab.

20. The apparatus of claim 22, wherein the protective sleeve is axially movable relative the brush head between a shielding position over the plurality of optic fibers and a retracted position exposing a sufficient length of the plurality of optic fibers to provide light and collect tissue samples.

21. The apparatus of claim 13, in combination with a power source and a processor.

22. The apparatus of claim 13, wherein the tissue swab includes a plurality of optic fibers.

23. (canceled)

24. The apparatus of claim 22, wherein the plurality of optic fibers additionally transmit light from the light source.

25. (canceled)

26. The apparatus of claim 13, further comprising a screw actuation mechanism disposed on the protective sleeve and sensor base.

27. The apparatus of any claim 26, wherein the screw actuation mechanism comprises a helical groove and a corresponding rail.

28.-51. (canceled)
